(19) United States
(12) Patent Application Publication
(10) Pub. No.: US 2005/0063949 A1
(43) Pub. Date: Mar. 24, 2005

(54) Method of treating hepatitis virus infection with a multiphasic interferon delivery profile

(76) Inventor: Gary Visor, Belmont, CA (US)

Correspondence Address: BOZICEVIC, FIELD & FRANCIS LLP, 1900 UNIVERSITY AVE, SUITE 200, EAST PALO ALTO, CA, 94303, US

(21) Appl. No.: 10/490,458

(22) PCT Filed: Sep. 26, 2002
(86) PCT No.: PCT/US02/30837

Related U.S. Application Data
(60) Provisional application No. 60/327,744, filed on Oct. 5, 2001.

(51) Int. Cl. A61K 38/21; A61K 9/22

(52) U.S. Cl. 424/85.7; 604/890.1

(57) ABSTRACT

The present invention provides methods of treating hepatitis virus infection. The methods generally involve administering a composition comprising an antiviral agent in a dosing regimen that achieves a multiphasic serum concentration profile of the antiviral agent. The dosing regiment includes dosing events that are less frequent than with currently available hepatitis therapies. The multiphasic antiviral agent serum concentration profile that is achieved using the methods of the invention effects an initial rapid drop in viral titer, followed by a further decrease in viral titer over time, to achieve a sustained viral response

### FIELD OF THE INVENTION

This invention is in the field of treatments for viral infections, in particular hepatitis virus.

### BACKGROUND OF THE INVENTION

Hepatitis C virus (HCV) infection is the most common chronic blood borne infection in the United States. Although the numbers of new infections have declined, the burden of chronic infection is substantial, with Centers for Disease Control estimates of 3.9 million (1.8%) infected persons in the United States. Chronic liver disease is the tenth leading cause of death among adults in the United States, and accounts for approximately 25,000 deaths annually, or approximately 1% of all deaths. Studies indicate that 40% of chronic liver disease is HCV-related, resulting in an estimated 8,000-10,000 deaths each year. HCV-associated end-stage liver disease is the most frequent indication for liver transplantation among adults.

Antiviral therapy of chronic hepatitis C has evolved rapidly over the last decade, with significant improvements seen in the efficacy of treatment. Nevertheless, even with combination therapy using pegylated IFN-α plus ribavirin, 40% to 50% of patients fail therapy, i.e., are nonresponders or relapsers. These patients currently have no effective therapeutic alternative. In particular, patients who have advanced fibrosis or cirrhosis on liver biopsy are at significant risk of developing complications of advanced liver disease, including ascites, jaundice, variceal bleeding, encephalopathy, and progressive liver failure, as well as a markedly increased risk of hepatocellular carcinoma.

The high prevalence of chronic HCV infection has important public health implications for the future burden of chronic liver disease in the United States. Data derived from the National Health and Nutrition Examination Survey (NHANES III) indicate that a large increase in the rate of new HCV infections occurred from the late 1960s to the early 1980s, particularly among persons between 20 to 40 years of age. It is estimated that the number of persons with long-standing HCV infection of 20 years or longer could more than quadruple from 1990 to 2015, from 750,000 to over 3 million. The proportional increase in persons infected for 30 or 40 years would be even greater. Since the risk of HCV-related chronic liver disease is related to the duration of infection, with the risk of cirrhosis progressively increasing for persons infected for longer than 20 years, this will result in a substantial increase in cirrhosis-related morbidity and mortality among patients infected between the years of 1965-1985.

Chronic hepatitis C virus infection is characterized by intermittent or persistent elevations in serum alanine aminotransferase (ALT) levels and constant levels of HCV RNA in the circulation. Currently, approved therapies use alpha interferons derived from natural leukocytes or by recombinant methods using cDNA sequences of specific subtypes or consensus interferon-α (IFN-α). The accepted dosage regimen is a subcutaneous administration of IFN-α in the dose ranges of 6-50 μg three times in week for a period of 24-48 weeks.

Cyclical administration of IFN-α has also been conducted, in the hope that viral clearance can be achieved. The repeat dosing has been deemed necessary in view of the rapid clearance and in vivo degradation of IFN-α. In another attempt to achieve better efficacy, combination therapies such as IFN-α and ribavirin have been carried out. In patients infected with the genotype 1 virus, which is the most prevalent HCV strain, only ≦25% of the patients demonstrated sustained viral response even with combination therapy. In attempts to improve further the therapeutic methods, various investigators have attempted a chemical modification of IFN-α by adding a polymer chain(s) to increase the molecular weight and size of the protein and to prolong the systemic circulation times. While these manipulations of IFN-α increased the circulation times and improved the efficacies further, a significant fraction of the protein loses its biological activity. Thus higher amounts of the protein have to be delivered to the patient with adverse effects such as neutropenia accompanying such administrations.

Viral kinetics during treatment regimens that include IFN-α have been examined. In general, an initial rapid decline in viral titers (early viral response; EVR) is seen in some individuals. The EVR results in an approximately 0.5- to 3-log decrease in serum HCV RNA levels in a period of 24-48 hours after initiation of treatment. An early robust response is favorable toward achieving a durable response. In some individuals, the EVR is followed by a further, less rapid decline of the virus in blood (second phase decline). The second phase decline is a slower decrease in the level of the virus over several weeks or months.

Despite the availability of approved treatment regimens discussed above, only a small fraction of the individuals treated attain a sustained viral response. Thus, there is a need in the art for improved methods for treating HCV infection. The present invention addresses this need.

Literature

U.S. Pat. Nos. 6,172,046; 6,245,740; 5,824,784; 5,372,808; 5,980,884; published international patent applications WO 96/21468; WO 96/11953; Torre et al. (2001) J. Med. Virol. 64:455-459; Bekkering et al. (2001) J. Hepatol. 34:435440; Zeuzem et al. (2001) Gastroenterol. 120:1438-1447; Zeuzem (1999) J. Hepatol. 31:61-64; Keeffe and Hollinger (1997) Hepatol. 26:101s-107S; Wills (1990) Clin. Pharmacokinet. 19:390-399; Heathcote et al. (2000) New Engl. J. Med. 343:1673-1680; Husa and Husova (2001) Bratisl. Lek. Listy 102:248-252; Glue et al. (2000) Clin. Pharmacol. 68:556-567; Bailon et al. (2001) Bioconj. Chem. 12:195-202; and Neumann et al. (2001) Science 282:103; Zalipsky (1995) Adv. Drug Delivery Reviews S. 16, 157-182; Mann et al. (2001) Lancet 358:958-965.

### SUMMARY OF THE INVENTION

The present invention provides methods of treating hepatitis virus infection. The methods generally involve administering a composition comprising an antiviral agent in a dosing regimen that achieves a multiphasic serum concentration profile of the antiviral agent. The dosing regimen includes dosing events that are less frequent than with currently available hepatitis therapies. The multiphasic antiviral agent serum concentration profile that is achieved using the methods of the invention effects an initial rapid drop in viral titer, followed by a further decrease in viral titer over time, to achieve a sustained viral response.

### FEATURES OF THE INVENTION

In some embodiments, the invention features a method for treating hepatitis C virus infection in an individual. The method generally involves administering a composition comprising interferon-α (IFN-α) in an amount effective to achieve a first serum concentration of IFN-α that is at least about 80% of the maximum tolerated dose (MTD) within a first period of time of about 24 to 48 hours, followed by a second concentration of IFN-α that is about 50% or less than the MTD, which second concentration is maintained for a second period of time of at least seven days. In some embodiments, a sustained viral response is achieved.

In some embodiments, the methods further include administering IFN-γ for a period of from about 1 day to about 14 days before administration of IFN-α.

In some embodiments, lFN-α is administered in a depot. In other embodiments, IFN-α is administered by continuous infusion. In some embodiments, continuous infusion administration is achieved with a pump. In other embodiments, IFN-α is administered by a single subcutaneous injection followed by continuous infusion using a pump.

In some embodiments, the invention features a method of treating hepatitis C virus infection in an individual, the method generally involving administering IFN-α in a dosing regimen comprising a first phase and a second phase, wherein, in the first phase, a first serum concentration of IFN-α is achieved that is at least about 80% of the maximum tolerated dose (MTD) within a first period of time of about 24 hours, wherein in the second phase, the ratio of the highest IFN-α serum concentration to the lowest serum IFN-α concentration, measured over any 24-hour period during the second phase, is less than 3, and wherein the highest concentration of IFN-α during the second phase is about 50% or less than the MTD. In some of these embodiments, the ratio of the highest IFN-α serum concentration to the lowest serum IFN-α concentration, measured over any 24-hour period during the second period of time is about 1.

In some embodiments, the invention features a method of treating hepatitis C virus infection in an individual, the method generally involving administering a composition comprising consensus interferon-α (CIFN) in an amount effective to achieve a first serum concentration of CIFN that is at least about 80% of the maximum tolerated dose (MTD) within a first period of time of about 24 hours, followed by a second concentration of CIFN that is about 50% or less than the MTD, which second concentration is maintained for a second period of time of at least seven days.

In some embodiments, the invention features a method of treating hepatitis C virus infection in an individual, the method generally involving administering consensus IFN-α (CIFN) in a dosing regimen comprising a first phase and a second phase, wherein, in the first phase, a first serum concentration of CIFN is achieved that is at least about 80% of the maximum tolerated dose (MTD) within a first period of time of about 24 hours, wherein in the second phase, the ratio of the highest CIFN serum concentration to the lowest serum CIFN concentration, measured over any 24-hour period during the second phase, is less than 3, and wherein the highest concentration of CIFN during the second phase is about 50% or less than the MTD.

In some embodiments, the invention features a method of treating hepatitis C virus infection in an individual, the method generally involving administering IFN-α in a dosing regimen comprising a first phase and a second phase, wherein, in the first phase, a first serum concentration C1max of IFN-α is achieved within a first period of time of about 24 hours, wherein in the second phase, a Csus is achieved that is about 50% of C1max or less, and wherein the area under the curve, defined by IFN-α serum concentration as a function of time, during any 24-hour time period in the second phase is no greater than the area under the curve of day 2 to day 3 as shown in FIG. 2.

In some embodiments, the invention features a method of treating hepatitis C virus infection in an individual, the method generally involving administering consensus IFN-α (CIFN) in a dosing regimen comprising a first phase and a second phase, wherein, in the first phase, a first serum concentration C1max of CIFN is achieved within a first period of time of about 24 hours, wherein in the second phase, a Csus is achieved that is about 50% of C1max or less, and wherein the area under the curve, defined by CIFN serum concentration as a function of time, during any 24-hour time period in the second phase is no greater than the area under the curve of day 2 to day 3 as shown in FIG. 2.

### BRIEF DESCRIPTION OF THE DRAWINGS

FIG. 1 is a graph depicting a viral kinetics during interferon-α therapy depicted here as clearance of HCV virus in blood as monitored by the level of viral RNA in serum using a sensitive measurement such as a polymerase chain reaction.

FIG. 2 is a graph depicting a profile of serum IFN-α concentration during administration of a controlled Release Injectible (CRI) system or a zero-order throughput system and bolus. Viral kinetics following conventional TIW regimen is included to contrast the improvements with a therapeutic dosing regimen according to the instant invention (see below). FIG. 3 is a graph depicting a profile of serum IFN-α concentration during administration of a controlled release injectible (CRI). In one scenario, the early phase concentration improves the initial viral decline (see dotted line).

FIG. 4 is a graph depicting the viral kinetics and pharmacokinetics following a CRI therapy. In this scenario, the early viral response (EVR) is similar to conventional TIW therapy. The high concentration Csus in second phase affects the slope, making the slope steep (see dotted line).

FIG. 5 is a graph depicting the viral kinetics and pharmacokinetics following a CRI therapy using IFN-α. In this scenario, there is significantly more decline in early viral titers and the second phase also exhibits a steep decline (see dotted line).

FIG. 6 is a graph depicting viral kinetics during administration of IFN-α with a sustained release delivery system providing repeat Cmax and Csus concentrations of drug to achieve significant sustained viral response. Because of repeat Cmaxs and sustained high Csus, a drop in viral titer can be seen as step (see dotted line).

### DEFINITIONS

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease (as in liver fibrosis that can result in the context of chronic HCV infection); (b) inhibiting the disease, i.e., arresting its development; and (c) relieving the disease, i.e., causing regression of the disease.

The terms “individual,” “host,” “subject,” and “patient” are used interchangeably herein, and refer to a mammal, including, but not limited to, primates, including simians and humans.

The term “early viral response (EVR),” used interchangeably with “initial viral response,” “rapid viral response” refers to the drop in viral titer within about 24 hours, about 48 hours, about 3 days, or about 1 week after the beginning of treatment for HCV infection.

The term “second phase decline” as used herein refers to a slower decrease in the level of the virus over several weeks or months after the EVR.

The term “sustained viral response” (SVR; also referred to as a “sustained response” or a “durable response”), as used herein, refers to the response of an individual to a treatment regimen for HCV infection, in terms of serum HCV titer. Generally, a “sustained viral response” refers to no detectable HCV RNA (e.g., less than about 500, less than about 200, or less than about 100 genome copies per milliliter serum) found in the patient's serum for a period of at least about one month, at least about two months, at least about three months, at least about four months, at least about five months, or at least about six months following cessation of treatment.

“Treatment failure patients” as used herein generally refers to HCV-infected patients who failed to respond to previous therapy for HCV (referred to as “non-responders”) or who initially responded to previous therapy, but in whom the therapeutic response was not maintained (referred to as “relapsers”). The previous therapy generally can include treatment with IFN-α monotherapy or IFN-α combination therapy, where the combination therapy may include administration of IFN-α and an antiviral agent such as ribavirin.

The term “hepatitis virus infection” refers to infection with one or more of hepatitis A, B, C, D, or E virus, with blood-borne hepatitis viral infection being of particular interest.

As used herein, the term “hepatic fibrosis,” used interchangeably herein with “liver fibrosis,” refers to the growth of scar tissue in the liver that can occur in the context of a chronic hepatitis infection.

As used herein, the term “liver function” refers to a normal function of the liver, including, but not limited to, a synthetic function, including, but not limited to, synthesis of proteins such as serum proteins (e.g., albumin, clotting factors, alkaline phosphatase, aminotransferases (e.g., alanine transaminase, aspartate transaminase), 5′-nucleosidase, γ-glutaminyltranspeptidase, etc.), synthesis of bilirubin, synthesis of cholesterol, and synthesis of bile acids; a liver metabolic function, including, but not limited to, carbohydrate metabolism, amino acid and ammonia metabolism, hormone metabolism, and lipid metabolism; detoxification of exogenous drugs; a hemodynamic function, including splanchnic and portal hemodynamics; and the like.

Drug delivery devices that are suitable for use in the subject methods include, but are not limited to, injection devices; an implantable device, e.g., pumps, such as an osmotic pump, that may or may not be connected to a catheter, biodegradable implants; liposomes; depots; and microspheres.

The term “dosing event” as used herein refers to administration of an antiviral agent to a patient in need thereof, which event may encompass one or more releases of an antiviral agent from a drug dispensing device. Thus, the term “dosing event,” as used herein, includes, but is not limited to, installation of a depot comprising an antiviral agent; installation of a continuous delivery device (e.g., a pump or other controlled release injectible system); and a single subcutaneous injection followed by installation of a continuous delivery system.

The term “depot” refers to any of a number of implantable, biodegradable or non-biodegradable, controlled release systems that are generally non-containerized and that act as a reservoir for a drug, and from which drug is released. Depots include polymeric non-polymeric biodegradable materials, and may be solid, semi-solid, or liquid in form.

The term “microsphere” (also referred to as “microparticles,” “nanospheres,” or “nanoparticles”) refers to small particles, generally prepared from a polymeric material and usually having a size in the range of from about 0.01 μm to about 0.1 μm, or from about 0.1 μm to about 10 μm in diameter.

The term “therapeutically effective amount” is meant an amount of a therapeutic agent, or a rate of delivery of a therapeutic agent, effective to facilitate a desired therapeutic effect. The precise desired therapeutic effect will vary according to the condition to be treated, the formulation to be administered, and a variety of other factors that are appreciated by those of ordinary skill in the art.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a dose” includes a plurality of such doses and reference to “the method” includes reference to one or more methods and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF THE INVENTION

The present invention provides methods of treating hepatitis virus infection, with hepatitis C virus (HCV) infection being of particular interest. The methods generally involve administering to an individual an antiviral agent in an amount effective to reduce the viral load in the individual, and in particular to achieve a sustained viral response in the individual. An antiviral agent is delivered to the individual in a dosing regimen that is effective to achieve a multiphasic concentration of the antiviral agent in the serum. The multiphasic concentration profile of the antiviral agent is designed to take into account the viral kinetics observed during treatment of hepatitis C virus (HCV) with IFN-α.

Currently available IFN-α therapies for treating HCV infection generally involve subcutaneous injections of IFN-α daily (QD), every other day (QOD), or three times a week (TIW).

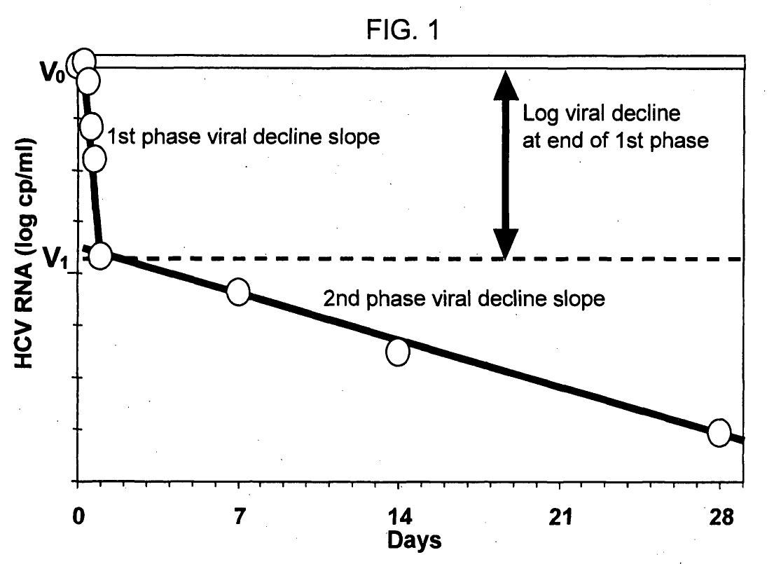

The kinetics of HCV infection among responders in response to conventional IFN-α therapies, as determined by RNA PCR, have been analyzed by mathematical modeling, and are shown in FIG. 1. Such studies have clearly shown a rapid viral decline phase in 2448 hours after the beginning of treatment, resulting in an approximately 0.5-log to an approximately. 3-log or greater decrease in serum RNA levels. This early viral response (EVR) is important in reducing the production of viral particles. An early, robust response is generally predictive of a more durable response. This early phase is usually followed by a slower, sustained clearance of the virus over several days or weeks.

Generally, this second phase is dependent on characteristics associated with the patient. Without wishing to be bound by any one theory, the second phase reduction in viral titer may be related to removal of virus-infected cells, e.g., by immune system mediated mechanisms. The slope of this second phase is determinative of the sustained viral response (SVR) of the patient, e.g., a steeper second phase slope is generally associated with a SVR and a positive treatment outcome.

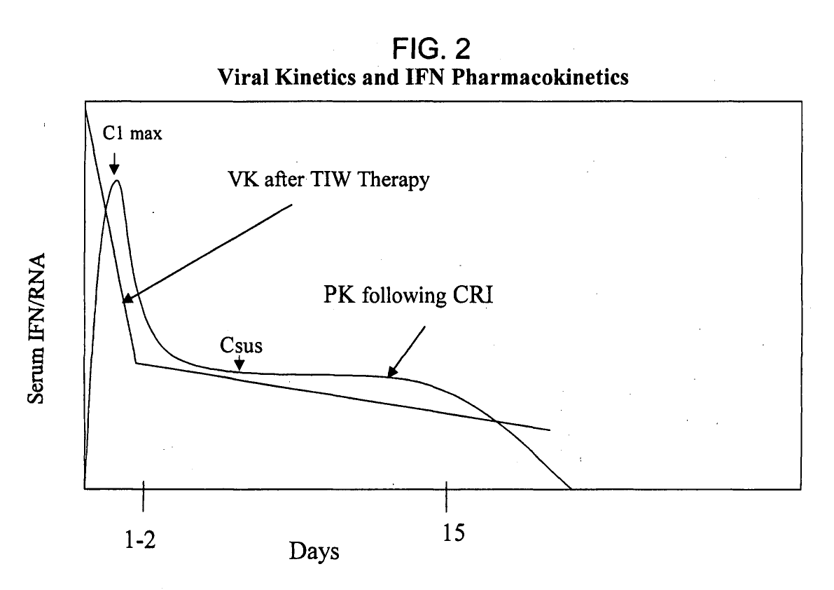

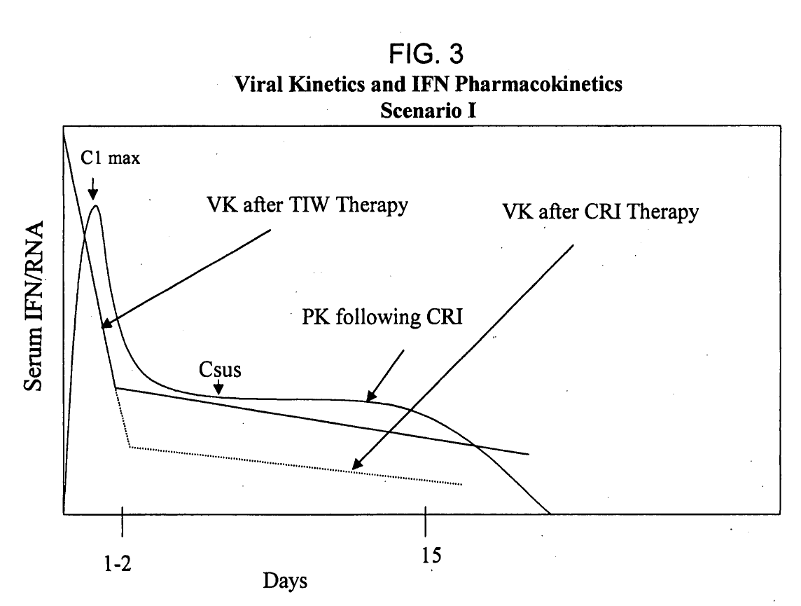

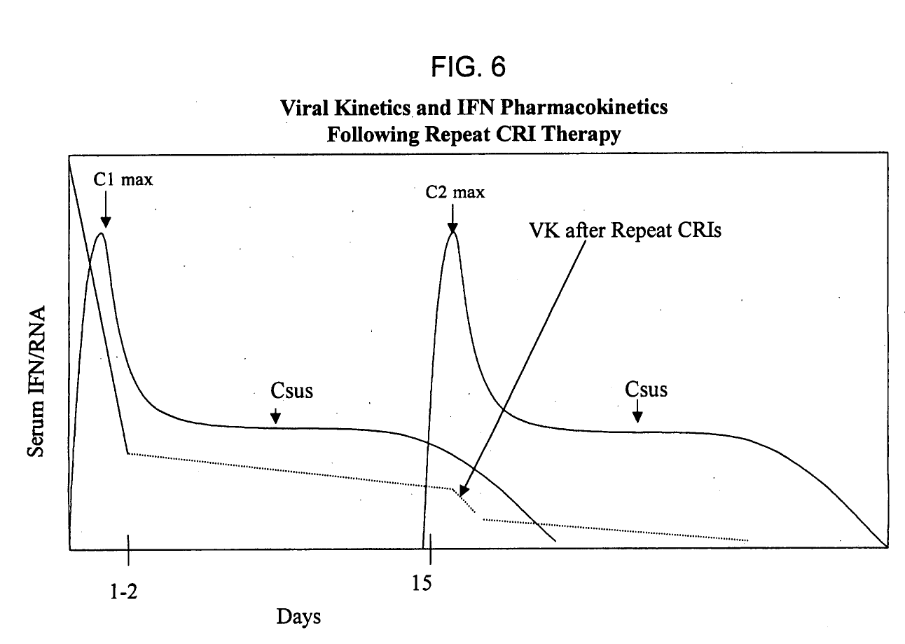

Viral kinetics and serum IFN-α concentration during the course of IFN-α-based therapy regimens are depicted in FIGS. 2-6. Viral kinetics (VK; depicted as viral RNA (“RNA”) versus time) is shown together with serum IFN-α concentration (pharmacokinetics (PK); depicted as serum IFN versus time) for both conventional (e.g., TIW) therapy and a therapeutic dosing regimen according to the invention (e.g., controlled release therapy, such as CRI therapy). The maximum serum concentrations of IFN-α achieved following pulsatile or repeat administrations of an active ingredient are denoted by C1max (FIGS. 2-5), C2max (FIG. 6), etc. C1max, C2max, etc., are at or near the maximum tolerated dose (MTD). The serum concentration of IFN-α achieved over a sustained period of time is denoted Csus (FIGS. 2-6). Csus is about 50% of the MTD. The amount of bioavailable antiviral drug is indicated by the area under the serum concentration versus time profile or area under the curve (AUC). The threshold concentration in serum when adverse effects appear is denoted the MTD.

Current therapies to treat HCV infection suffer from certain drawbacks. Dosing regimens involving daily (QD), every other day (QOD), or thrice weekly (TIW) injections of IFN-α over extended treatment periods suffer from one or more of the following drawbacks: (1) the dosing regimens are uncomfortable to the patient and, in some cases, result in reduced patient compliance; (2) the dosing regimens are often associated with adverse effects, causing additional discomfort to the patient, and, in some cases, resulting in reduced patient compliance; (3) the dosing regimens result in “peaks” (Cmax) and “troughs” (Cmin) in serum IFN-α concentration, and, during the “trough” periods, virus can replicate, and/or infect additional cells, and/or mutate; (4) in many cases, the log reduction in viral titer during the early viral response is insufficient to effect a sustained viral response that ultimately results in clearance of the virus (see FIG. 2; viral kinetics after conventional IFN-α TIW therapy).

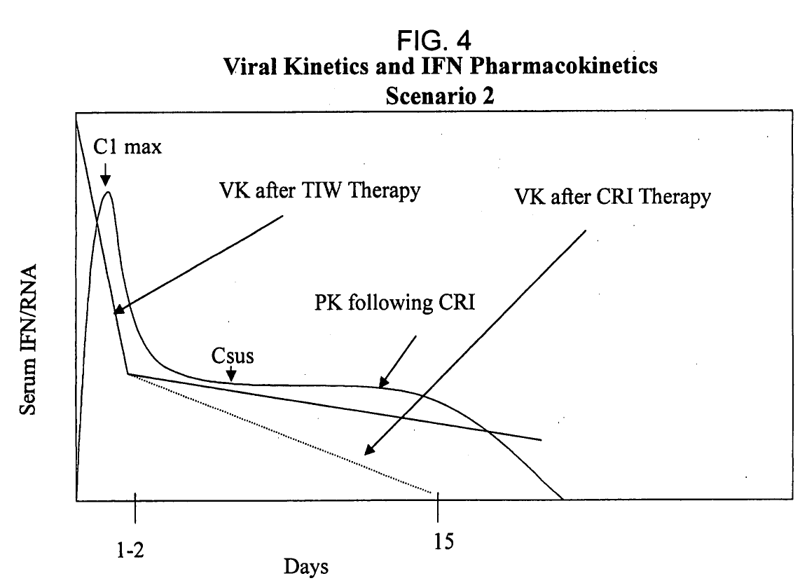

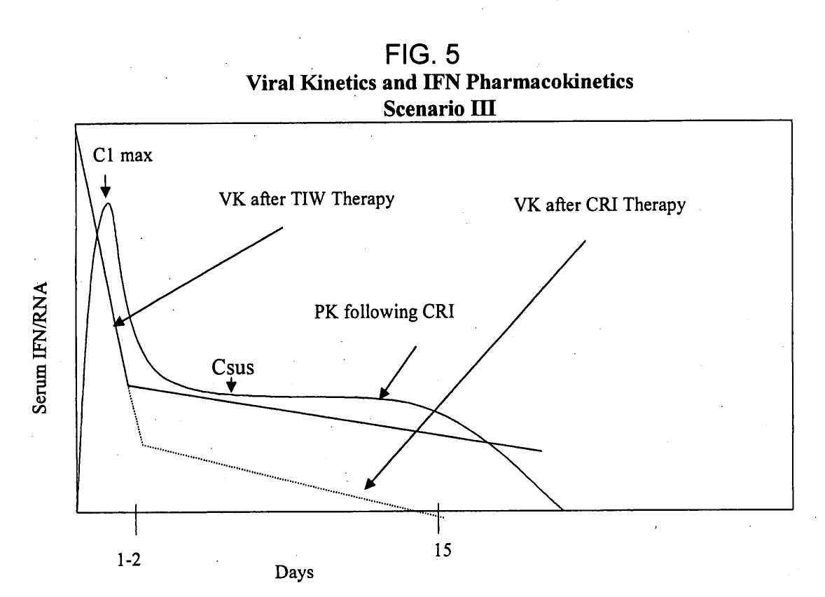

The instant invention provides dosing regimens that avoids these drawbacks, and provides significant advantages, including the following: (1) because the administration is less frequent than QD, QOD, or TIW, patient discomfort is reduced, which potentially increases patient compliance; (2) because the dosing is continuous over a period of time, “peaks” (i.e., Cmax) and “toughs” (i.e., Cmin) in serum IFN-α concentrations are avoided, e.g., the Cmax to Cmin ratio is reduced; (3) because the peak/trough cycles associated with previous dosing regimens are avoided, adverse effects are reduced; (4) because the peak/trough cycles associated with previous dosing regimens are avoided, viral replication, infection of further cells, and mutation is reduced (i.e., there is constant “pressure” on the virus, as there is a more constant level of antiviral agent in the serum); (5) one dosing event according to the invention addresses both the early viral response and the sustained viral responses phases of viral kinetics (see, e.g., FIG. 5, scenario III); (6) repeated dosing events according to the invention has an effect on the sustained viral response, reducing viral titer still further (see, e.g., FIG. 6: C1max, C2max, etc., exert enormous negative selective pressure on the virus, reducing viral mutation and/or replication and/or evasion events between dosing cycles); (7) the log reduction in viral titer during first phase of the dosing event according to the invention is greater than with previously available dosing regimens discussed above (see, e.g., FIG. 3, scenario 1; (8) the constant high drug concentration in the sustained phase (Csus) makes the second phase slope steeper (see, e.g., FIG. 4, scenario H); and (9) because the log reduction in viral titer is increased, the outcome during the second phase is more favorable, i.e., the decrease in the viral titer during the sustained viral response phase is more rapid (the slope is steeper) than with previous dosing regimens discussed above.

The present invention provides methods of treating hepatitis viral infection, involving a dosing regimen that provides for a multiphasic serum concentration of antiviral agent. The multiphasic serum concentration of antiviral agent is achieved with less frequent dosing events than with current therapies.

During a first phase, the serum concentration of IFN-α is high, to provide optimal Cmax concentrations, and to effect as steep a slope as possible in the viral titer, bringing the viral titer down rapidly such that a lower concentration of IFN-α will be effective. This initial high dose of IFN-α is referred to as the “first dose” or the “initial loading dose”.

During a second phase, the IFN-α serum concentration is lower than in the first phase, and is effective to reduce the viral titer still further. The first phase is kept as short as possible, since the amount of IFN-α delivered during this phase is at or near the maximum dose that is tolerated by an individual (the “MTD”). Once the viral titer is brought down quickly during this initial, high-dosage phase, the concentration of IFN-α can be lowered, yet achieve an AUC sufficient remain effective to reduce still further the viral titer (see, e.g., FIG. 3). This second, lower concentration of IFN-α is tolerated by most individuals; thus, patient comfort and compliance is maximized.

The first phase and the second phase are achieved in a single dosing event, e.g., where a “single dosing event” includes installation of a depot; installation of a pump; and the combination of a single subcutaneous injection followed by installation of a pump. A single dosing event is achieved by one or more dosage forms, e.g., one or more of: a depot; a pump; and an injection device.

In some embodiments, antiviral agent is administered in a depot. This form of administration takes advantage of a property of depot delivery that is generally considered undesirable, namely the initial “burst” of drug release from the depot after implantation or injection into a patient. By delivering the antiviral agent in a depot formulation that does release an initial burst of antiviral agent, a multiphasic serum concentration of antiviral agent is achieved. The initial burst of antiviral agent release effects the first serum concentration of antiviral agent that is effective in bringing down viral titers quickly, to a level that is treatable by a lower concentration of antiviral agent. This lower serum concentration of antiviral agent is achieved by the sustained release of antiviral agent from the depot following the initial burst.

In many embodiments, the dosing regimen involves a single dosing event. In other embodiments, the dosing regimen dosing event is repeated. Repeat administrations using such delivery systems provide C1max, C2max, etc., in each case followed by a steady state concentration (Csus; as shown in FIG. 6).

Methods of Treating a Hepatitis Infection

The instant invention provides methods of treating a hepatitis virus infection. The methods generally involve administering an antiviral agent at a level and in a manner effective to achieve a multiphasic serum concentration of the antiviral agent. A first phase and a second phase are achieved with a single dosing event (e.g., installation (e.g., implantation or injection) of a depot; installation of a continuous infusion device, such as a pump; a combination of a single subcutaneous injection and installation of a continuous infusion device).

In all embodiments of the invention, the dosing regimens of the methods of the invention achieve serum concentrations of antiviral agent in which the “peaks” (Cmax; the highest serum concentration of antiviral agent) and “troughs” (Cmin; the lowest serum concentration of antiviral agent) of serum antiviral agent concentration are reduced or avoided. In all embodiments, the dosing regimens of the instant methods result-in Cmax:Cmin ratio of less than about 3.0, less than about 2.5, less than about 2.0, or less than about 1.5 during the second phase (e.g., during days 2-15 of treatment, during days 2-10 of treatment, during days 3-10 of treatment, or during days 3-15 of treatment, as shown in FIGS. 2-6). In some embodiments, the dosing regimens achieve a Cmax:Cmin ratio of about 1.0 during the second phase (e.g., during days 2-15 of treatment, during days 2-10 of treatment, during days 3-10 of treatment, or during days 3-15 of treatment, as shown in FIGS. 2-6).

In general, in the dosing regimens of the methods of the invention, an area under the curve (AUC) of antiviral agent serum concentration versus time during the second phase, measured during any 24-hour period of the second phase, (i.e., AUCₛᵤₛ is less than the AUC for any 24-hours period of the first phase (i.e., AUCₘₐₓ). In other words, the AUCₛᵤₛ measured during any 24-hour period of the second phase is less than the AUCₘₐₓ measured during any 24-hour period of the first phase.

The serum concentration of antiviral agent in the first phase is effective to achieve a 1.5-log, a 2-log, a 2.5-log, a 3-log, a 3.5-log, a 4-log, a 4.5-log, or a 5-log reduction in viral titer in the serum of the individual.

The serum concentration of antiviral agent in the first phase is effective to achieve a 1.5-log, a 2-log, a 2.5-log, a 3-log, a 3.5-log, a 4-log, a 4.5-log, or a 5-log reduction in viral titer in the serum of the individual within a period of from about 12 hours to about 48 hours, or from about 16 hours to about 24 hours after the beginning of the dosing regimen.

The second concentration of antiviral agent is maintained for a period of from about 24 hours to about 48 hours, from about 2 days to about 4 days, from about 4 days to about 7 days, from about 1 week to about 2 weeks, from about 2 weeks to about 4 weeks, from about 4 weeks to about 6 weeks, from about 6 weeks to about 8 weeks, from about 8 weeks to about 12 weeks, from about 12 weeks to about 16 weeks, from about 16 weeks to about 24 weeks, or from about 24 weeks to about 48 weeks.

In the second phase, the concentration of antiviral agent in the serum is effective to reduce viral titers to undetectable levels, e.g., to about 1000 to about 5000, to about 500 to about 1000, or to about 100 to about 500 genome copies/mL serum. In some embodiments, an effective amount of antiviral agent is an amount that is effective to reduce viral load to lower than 100 genome copies/mL serum.

The serum concentration of antiviral agent in the second phase is effective to achieve a sustained viral response, e.g., no detectable HCV RNA (e.g., less than about 500, less than about 200, or less than about-100 genome copies per milliliter serum) is found in the patient's serum for a period of at least about one month, at least about two months, at least about three months, at least about four months, at least about five months, or at least about six months following cessation of therapy.

In some embodiments, at least a third phase follows the first and second phases. In some of these embodiments, third phase includes administering antiviral agent in a dose effective to achieve a serum concentration of antiviral agent equal or nearly equal to that of the first serum concentration. In some of these embodiments, a fourth phase includes administering antiviral agent in a dose effective to achieve a serum concentration of antiviral agent equal or nearly equal to that of the second serum concentration.

IFN-α Treatment of HCV Infection

In certain embodiments of interest, the hepatitis virus is hepatitis C virus (HCV). In particular embodiments of interest, the hepatitis virus is HCV, and the antiviral agent is interferon-α (IFN-α).

In a first phase, a serum concentration of IFN-α is achieved that is at or near the maximum level that is tolerable by the patient. The serum concentration that is achieved in the first phase (the first concentration) is in a range of from about 10 to about 1000, from about 10 to about 500, from about 20 to about 250, from about 30 to about 100, or from about 50 to about 75 International Units (IU)/ml. The first serum concentration is maintained for a period of from about 6 hours to about 12 hours, from about 12 hours to about 24 hours, or from about 24 hours to about 48 hours.

In the first phase, an amount of IFN-α is administered that is effective to achieve a serum concentration of IFN-α that is from about 65% to about 70%, from about 70% to about 75%, from about 75% to about 80%, from about 80% to about 85%, from about 85% to about 90%, from about 90% to about 95%, or from about 95% to about 100% of the maximum tolerated dose (MTD). Thus, within a period of from about 6 hours to about 12 hours, from about 12 hours to about 24 hours, or from about 24 hours to about 48 hours from the beginning of the dosing regimen, a serum concentration of IFN-α is achieved that is from about 65% to about 70%, from about 70% to about 75%, from about 75% to about 80%, from about 80% to about 85%, from about 85% to about 90%/, from about 90% to about 95%, or from about 95% to about 100% of the maximum tolerated dose (MTD).

The administered dose to achieve the first serum concentration of IFN-α is in a range of from about 10 μg to about 100 μg, from about 20 μg to about 70 μg, from about 25 μg to about 60 μg, from about 30 μg to about 50 μg. These various doses refer to free interferon and the amounts of the depots to administer to achieve this will depend on drug loading efficiencies, as discussed below.

Effective dosages of consensus IFN-α include about 3 μg, about 9 μg, about 15 μg, about 18 μg, or about 27 μg per dose. Effective dosages of IFN-α2a and IFN-α2b range from 3 million international units (MIU) to 10 MIU per dose. Effective dosages of PEGylated IFN-α2a range from 90 to 180 μg per dose. Effective dosages of PEGylated IFN-α2b range from 0.5 μg/kg body weight to 1.5 μg/kg body weight per dose.

Patients with chronic hepatitis C generally have circulating virus at levels of 10⁵-10⁷ genome copies/ml. In this first phase, the serum concentration of IFN-α is effective to reduce HCV titer down to about 5×10⁴ to about 10⁵, to about 10⁴ to about 5×10⁴, or to about 5×10³ to about 10⁴ genome copies per milliliter serum.

In some embodiments, the serum concentration of IFN-α in the first phase is effective to reduce HCV titer down to about 5×10⁴ to about 10⁵, to about 10⁴ to about 5×10⁴, or to about 5×10³ to about 10⁴ genome copies per milliliter serum within a period of from about 12 hours to about 48 hours, or from about 16 hours to about 24 hours after the beginning of the dosing regimen.

In some embodiments, the serum concentration of IFN-α in the first phase is effective to achieve a 1.5-log, a 2-log, a 2.5-log, a 3-log, a 3.5-log, a 4-log, a 4.5-log, or a 5-log reduction in viral titer in the serum of the individual.

In some embodiments, the serum concentration of IFN-α in the first phase is effective to achieve a 1.5-log, a 2-log, a 2.5-log, a 3-log, a 3.5-log, a 4-log, a 4.5-log, or a 5-log reduction in viral titer in the serum of the individual within a period of from about 12 hours to about 48 hours, or from about 16 hours to about 24 hours after the beginning of the dosing regimen.

In the first phase, a serum concentration of IFN-α is achieved that is effective to reduce the viral titer to a level that is treatable with a dose of interferon that can be tolerated by an infected individual.

In the second phase, IFN-α is administered at a level that is effective to achieve a serum concentration of IFN-α that is well below the maximum level that can be tolerated by the patient, and that is effective to reduce the viral titer still further. In the second phase, IFN-α is administered at a dose that is effective to achieve a serum concentration of IFN-α of from about 5 IU/ml to about 50 IU/ml. In some embodiments, IFN-α is administered at a dose that is effective to achieve a serum concentration of IFN-α of from about 5 IU/ml to about 100 IU/ml or higher. In this second phase, the administered dose of IFN-α is in a range of from about 0.5×10⁶ IU to about 50×10⁶ IU.

In the second phase, IFN-α is administered at a level that is effective to achieve and maintain a serum concentration of IFN-α that is from about 10% to about 15%, from about 15% to about 20%, from about 20% to about 25%, from about 25% to about 30%, from about 30% to about 35%, from about 35% to about 40%, from about 40% to about 45%, or from about 45% to about 50% of the MTD. The serum concentration of IFN-α in the second phase is well below the MTD, yet effective to exert and antiviral effect. Thus, over a period of from about 48 hours to about 4 days, from about 48 hours to about 7 days, from about 48 hours to about 10 days, or from about 48 hours to about 15 days, after the beginning of the dosing regimen, a serum concentration of IFN-α is achieved (and generally maintained) that is from about 10% to about 15%, from about 15% to about 20%, from about 20% to about 25%, from about 25% to about 30%, from about 30% to about 35%, from about 35% to about 40%, from about 40% to about 45%, or from about 45% to about 50% of the MTD.

The second concentration of IFN-α is maintained for a period of from about 24 hours to about 48 hours, from about 2 days to about 4 days, from about 4 days to about 7 days, from about 1 week to about 2 weeks, from about 2 weeks to about 4 weeks, from about 4 weeks to about 6 weeks, from about 6 weeks to about 8 weeks, from about 8 weeks to about 12 weeks, from about 12 weeks to about 16 weeks, from about 16 weeks to about 24 weeks, or from about 24 weeks to about 48 weeks.

In the second phase, the second concentration of serum IFN-α is effective to reduce viral titers to about 1000 to about 5000, to about 500 to about 1000, or to about 100 to about 500 genome copies/mL serum. In some embodiments, an effective amount of IFNα is an amount that is effective to reduce viral load to lower than 100 genome copies/mL serum.

The second concentration of serum IFN-α is effective to achieve a sustained viral response, e.g., no detectable HCV RNA (e.g., less than about 500, less than about 200, or less than about 100 genome copies per milliliter serum) is found in the patient's serum for a period of at least about one month, at least about two months, at least about three months, at least about four months, at least about five months, or at least about six months following cessation of therapy.

In some embodiments, at least a third phase follows the first and second phases. In some of these embodiments, third phase includes administering IFN-α in a dose effective to achieve a serum concentration of IFN-α equal or nearly equal to that of the first serum concentration. In some of these embodiments, a fourth phase includes administering IFN-α in a dose effective to achieve a serum concentration of IFN-α equal or nearly equal to that of the second serum concentration.

Combination Therapies

In some embodiments, the methods provide for combination therapy comprising administering IFN-α and an additional therapeutic agent such as IFN-γ and/or ribavirin. In all embodiments in which the dosing regimen comprises administration of IFN-α and an additional agent such as IFN-γ and/or ribavirin, IFN-α is administered such that a multiphasic serum concentration of IFN-α is achieved, as described above.

In some embodiments, the additional therapeutic agent(s) is administered during the entire course of IFN-α treatment, and the beginning and end of the treatment periods coincide. In other embodiments, the additional therapeutic agent(s) is administered for a period of time that is overlapping with that of the IFN-α treatment, e.g., treatment with the additional therapeutic agent(s) begins before the IFN-α treatment begins and ends before the IFN-α treatment ends; treatment with the additional therapeutic agent(s) begins after the IFN-α treatment begins and ends after the IFN-γ treatment ends; treatment with the additional therapeutic agent(s) begins after the IFN-α treatment begins and ends before the IFN-α treatment ends; or treatment with the additional therapeutic agent(s) begins before the IFN-α treatment begins and ends after the IFN-α treatment ends.

In still other embodiments, the additional therapeutic agent(s) is administered before the IFN-α treatment begins, and ends once IFN-α treatment begins, e.g., the additional therapeutic agent is used in a “priming” dosing regimen.

IFN-α and IFN-γ

In some embodiments, interferon gamma (IFN-γ) is administered separately from IFN-α, e.g., the IFN-γ is administered in a separate formulation and in a separate dosing event from IFN-α. In other embodiments, IFN-γ is administered in the same formulation with IFN-α (and therefore in the same dosing event). In still other embodiments, IFN-γ is administered in a separate formulation from IFN-α, and is administered in a dosing regimen that provides for a multiphasic serum concentration as described above.

Effective dosages of IFN-γ range from about 0.5 μg/m² to about 500 μg/m², usually from about 1.5 μg/m² to 200 μg/m², depending on the size of the patient. This activity is based on 10⁶ international units (IU) per 50 μg of protein.

As noted above, in some embodiments, IFN-γ is administered in a separate dosing event, as IFN-α. In one non-limiting example, IFN-γ is administered in a dose of about 1 MIU/day for 14 days; followed by 5 MIU/day for 14 days; followed by 5MIU three times per week for 22 weeks.

In some embodiments, IFN-γ is administered during the entire course of IFN-α treatment. In other embodiments, IFN-γ is administered for a period of time that is overlapping with that of the IFN-α treatment, e.g., the IFN-γ treatment can begin before the IFN-α treatment begins and end before the IFN-α treatment ends; the IFN-γ treatment can begin after the IFN-α treatment begins and end after the IFN-γ treatment ends; the IFN-γ treatment can begin after the IFN-α treatment begins and end before the IFN-α treatment ends; or the IFN-γ treatment can begin before the IFN-α treatment begins and end after the IFN-α treatment ends.

In some embodiments, IFN-γ is administered for a period of time before IFN-α is administered. Without wishing to be bound by any one theory, IFN-γ may effect a Th2 to Th1 shift. This increase in a Th1 immune response may result in an increase in the rate of reduction of viral titer once IFN-α administration is initiated. In these embodiments, IFN-γ is administered for a period of time from about 1 day to about 14 days, from about 2 days to about 10 days, or from about 3 days to about 7 days, before the beginning of treatment with IFN-α. This period of time is referred to as the “priming” phase. In some of these embodiments, IFN-γ treatment is continued throughout the entire period of treatment with IFN-α. In other embodiments, IFN-γ treatment is discontinued before the end of treatment with IFN-α. In these embodiments, the total time of treatment with IFN-γ (including the “priming” phase) is from about 2 days to about 30 days, from about 4 days to about 25 days, from about 8 days to about 20 days, from about 10 days to about 18 days, or from about 12 days to about 16 days.

IFN-γ can be administered by any conventional route and means, including, but not limited to, subcutaneously, intradermally, orallyl etc. IFN-γ can also be administered by the methods of the invention, providing for multiphasic serum concentration of IFN-γ. Administration can be by injection, by a continuous infusion device (e.g., a pump), and the like. In many embodiments, IFN-γ is administered subcutaneously by injection.

IFN-α and Ribavirin

Ribavirin, 1-β-D-ribofuranosyl-1H-1,2,4-triazole-3-carboxamide, available from ICN Pharmaceuticals, Inc., Costa Mesa, Calif., is described in the Merck Index, compound No. 8199, Eleventh Edition. Its manufacture and formulation is described in U.S. Pat. No. 4,211,771. The invention also contemplates use of derivatives of ribavirin (see, e.g., U.S. Pat. No. 6,277,830). The ribavirin may be administered orally in capsule or tablet form, or in the same or different administration form and in the same or different route as the IFN-α. Of course, other types of administration of both medicaments, as they become available are contemplated, such as by nasal spray, transdermally, intravenously, by suppository, by sustained release dosage form, etc. Any form of administration will work so long as the proper dosages are delivered without destroying the active ingredient.

Ribavirin is generally administered in an amount ranging from about 30 mg to about 60 mg, from about 60 mg to about 125 mg, from about 125 mg to about 200 mg, from about 200 mg to about 300 gm, from about 300 mg to about 400 mg, from about 400 mg to about 1200 mg, from about 600 mg to about 1000 mg, or from about 700 to about 900 mg per day.

In some embodiments, ribavirin is administered throughout the entire course of IFN-α treatment. In other embodiments, ribavirin is administered less than the entire course of IFN-α treatment, e.g., only during the first phase of IFN-α treatment, only during the second phase of IFN-α treatment, or some other portion of the IFN-α treatment regimen.

Antiviral Agents

Any of a variety of antiviral agents can be delivered using the methods of the invention. Antiviral agents suitable for use in the instant methods include, but are not limited to, IFN-α, IFN-γ, and ribavirin.

IFN-Alpha

Any known IFN-α can be used in the instant invention. The term “interferon-alpha” as used herein refers to a family of related polypeptides that inhibit viral replication and cellular proliferation and modulate immune response. The term “IFN-α” includes naturally occurring IFN-α; synthetic IFN-α; derivatized IFN-α (e.g., PEGylated IFN-α, glycosylated IFN-α, and the like); and analogs of naturally occurring or synthetic IFN-α; essentially any IFN-α that has antiviral properties, as described for naturally occurring IFN-α.

Suitable alpha interferons include, but are not limited to, naturally-occurring IFN-α (including, but not limited to, naturally occurring IFN-α2a, IFN-α2b); recombinant interferon alpha-2b such as Intron®A interferon available from Schering Corporation, Kenilworth, N.J.; recombinant interferon alpha-2a such as Roferon® interferon available from Hoffmann-La Roche, Nutley, N.J.; recombinant interferon alpha-2C such as Berofor® alpha 2 interferon available from Boehringer Ingelheim Pharmaceutical, Inc., Ridgefield, Conn.; interferon alpha-n1, a purified blend of natural alpha interferons such as Sumiferon available from Sumitomo, Japan or as Wellferon® interferon alpha-n1 (INS) available from the Glaxo-Wellcome Ltd., London, Great Britain; and interferon alpha-n3 a mixture of natural alpha interferons made by Interferon Sciences and available from the Purdue Frederick Co., Norwalk, Conn., under the Alferon® Tradename.

The term “IFN-α” also encompasses consensus IFN-α. Consensus IFN-α (also referred to as “CIFN” and “IFN-con”) encompasses but is not limited to the amino acid sequences designated IFN-con₁, IFN-con₂ and IFN-con₃ which are disclosed in U.S. Pat. Nos. 4,695,623 and 4,897,471; and consensus interferon as defined by determination of a consensus sequence of naturally occurring interferon alphas (e.g., Infergen®, Amgen, Thousand Oaks, Calif.). DNA sequences encoding IFN-con may be synthesized as described in the aforementioned patents or other standard methods. Use of CIFN is of particular interest.

The term “IFN-α” also encompasses derivatives of IFN-α that are derivatized (e.g., are chemically modified) to alter certain properties such as serum half-life. As such, the term “IFN-α” includes glycosylated IFN-α; IFN-α derivatized with polyethylene glycol (“PEGylated IFN-α”); and the like. PEGylated IFN-α, and methods for making same, is discussed in, e.g., U.S. Pat. Nos. 5,382,657; 5,981,709; and 5,951,974. PEGylated IFN-α encompasses conjugates of PEG and any of the above-described IFN-α molecules, including, but not limited to, PEG conjugated to interferon alpha-2a (Roferon, Hoffman La-Roche, Nutley, N.J.), interferon alpha 2b (Intron, Schering-Plough, Madison, N.J.), interferon alpha-2c (Berofor Alpha, Boehringer Ingelheim, Ingelheim, Germany); and consensus interferon as defined by determination of a consensus sequence of naturally occurring interferon alphas (infergen, Amgen, Thousand Oaks, Calif.).

Interferon-Gamma

The nucleic acid sequences encoding IFN-γ polypeptides may be accessed from public databases, e.g., Genbank, journal publications, etc. While various mammalian IFN-γ polypeptides are of interest, for the treatment of human disease, generally the human protein will be used. Human IFN-γ coding sequence may be found in Genbank, accession numbers X13274; V00543; and NM_{—}000619. The corresponding genomic sequence may be found in Genbank, accession numbers J00219; M37265; and V00536. See, for example. Gray et al. (1982) Nature 295:501 (Genbank X13274); and Rinderknecht et al. (1984) J.B.C. 259:6790.

IFN-γ1b (Actimmune®; human interferon) is a single-chain polypeptide of 140 amino acids. It is made recombinantly in E.coli and is unglycosylated. Rinderknecht et al. (1984) J. Biol. Chem. 259:6790-6797.

The IFN-γ to be used in the methods of the present invention may be any of natural IFN-γs, recombinant IFN-γs and the derivatives thereof so far as they have an IFN-γ activity, particularly human IFN-γ activity. Human IFN-γ exhibits the antiviral and anti-proliferative properties characteristic of the interferons, as well as a number of other immunomodulatory activities, as is known in the art. Although IFN-γ is based on the sequences as provided above, the production of the protein and proteolytic processing can result in processing variants thereof. The unprocessed sequence provided by Gray et al., supra, consists of 166 amino acids (aa). Although the recombinant IFN-γ produced in E. coli was originally believed to be 146 amino acids, (commencing at amino acid 20) it was subsequently found that native human IFN-1 is cleaved after residue 23, to produce a 143 aa protein, or 144 aa if the terminal methionine is present, as required for expression in bacteria. During purification, the mature protein can additionally be cleaved at the C terminus after reside 162 (referring to the Gray et al. sequence), resulting in a protein of 139 amino acids, or 140 amino acids if the initial methionine is present, e.g. if required for bacterial expression. The N-terminal methionine is an artifact encoded by the mRNA translational “start” signal AUG that, in the particular case of E. coli expression is not processed away. In other microbial systems or eukaryotic expression systems, methionine may be removed.

For use in the subject methods, any of the native IFN-γ peptides, modifications and variants thereof, or a combination of one or more peptides may be used. IFN-γ peptides of interest include fragments, and can be variously truncated at the carboxy terminal end relative to the full sequence. Such fragments continue to exhibit the characteristic properties of human gamma interferon, so long as amino acids 24 to about 149 (numbering from the residues of the unprocessed polypeptide) are present. Extraneous sequences can be substituted for the amino acid sequence following amino acid 155 without loss of activity. See, for example, U.S. Pat. No. 5,690,925. Native IFN-γ moieties include molecules variously extending from amino acid residues 24-150; 24-151, 24-152; 24-153, 24-155; and 24-157. Any of these variants, and other variants known in the art and having IFN-γ activity, may be used in the present methods.

The sequence of the IFN-γ polypeptide may be altered in various ways known in the art to generate targeted changes in sequence. A variant polypeptide will usually be substantially similar to the sequences provided herein, i.e., will differ by at least one amino acid, and may differ by at least two but not more than about ten amino acids. The sequence changes may be substitutions, insertions or deletions. Scanning mutations that systematically introduce alanine, or other residues, may be used to determine key amino acids. Specific amino acid substitutions of interest include conservative and non-conservative changes. Conservative amino acid substitutions typically include substitutions within the following groups: (glycine, alanine); (valine, isoleucine, leucine); (aspartic acid, glutamic acid); (asparagine, glutamine); (serine, threonine); (lysine, arginine); or (phenylalanine, tyrosine).

Modifications of interest that may or may not alter the primary amino acid sequence include chemical derivatization of polypeptides, e.g., acetylation, or carboxylation; changes in amino acid sequence that introduce or remove a glycosylation site; changes in amino acid sequence that make the protein susceptible to PEGylation; and the like. Also included are modifications of glycosylation, e.g., those made by modifying the glycosylation patterns of a polypeptide during its synthesis and processing or in further processing steps; e.g., by exposing the polypeptide to enzymes that affect glycosylation, such as mammalian glycosylating or deglycosylating enzymes. Also embraced are sequences that have phosphorylated amino acid residues, e.g., phosphotyrosine, phosphoserine, or phosphothreonine.

Included in the subject invention are polypeptides that have been modified using ordinary chemical techniques so as to improve their resistance to proteolytic degradation, to optimize solubility properties, or to render them more suitable as a therapeutic agent. For examples, the backbone of the peptide may be cyclized to enhance stability (see Friedler et al. (2000) J. Biol. Chem. 275:23783-23789). Analogs may be used that include residues other than naturally occurring L-amino acids, e.g., D-amino acids or non-naturally occurring synthetic amino acids. The protein may be pegylated to enhance stability.

The polypeptides may be prepared by in vitro synthesis, using conventional methods as known in the art, by recombinant methods, or may be isolated from cells induced or naturally producing the protein. The particular sequence and the manner of preparation will be determined by convenience, economics, purity required, and the like. If desired, various groups may be introduced into the polypeptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

The polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

Ribavirin

Ribavirin, 1-β-D-ribofuranosyl-1H-1,2,4-triazole-3-carboxamide, available from ICN Pharmaceuticals, Inc., Costa Mesa, Calif., is described in the Merck Index, compound No. 8199, Eleventh Edition. Its manufacture and formulation is described in U.S. Pat. No. 4,211,771. The invention also contemplates use of derivatives of ribavirin (see, e.g., U.S. Pat. No. 6,277,830).

Liver Targeting Systems

Antiviral agents described herein can be targeted to the liver, using any known targeting means. Those skilled in the art are aware of a wide variety of compounds that have been demonstrated to target compounds to hepatocytes. Such liver targeting compounds include, but are not limited to, asialoglycopeptides; basic polyamino acids conjugated with galactose or lactose residues; galactosylated albumin; asialoglycoprotein-poly-L-lysine) conjugates; lactosaminated albumin; lactosylated albumin-poly-L-lysine conjugates; galactosylated poly-L-lysine; galactose-PEG-poly-L-lysine conjugates; lactose-PEG-poly-L-lysine conjugates; asialofetuin; and lactosylated albumin.

In some embodiments, a liver targeting compound is conjugated directly to the antiviral agent. In other embodiments, a liver targeting compound is conjugated indirectly to the antiviral agent, e.g., via a linker. In still other embodiments, a liver targeting compound is associated with a delivery vehicle, e.g., a liposome or a microsphere, forming a hepatocyte targeted delivery vehicle, and the antiviral agent is delivered using the hepatocyte targeted delivery vehicle.

The terms “targeting to the liver” and “hepatocyte targeted” refer to targeting of an antiviral agent to a hepatocyte, such that at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, or more, of the antiviral agent administered to the subject enters the liver via the hepatic portal and becomes associated with (e.g., is taken up by) a hepatocyte.

Drug Delivery Systems

Any known delivery system that is capable of providing a multiphasic serum concentration profile of antiviral agent can be used in the present invention. In addition, a combination of any known delivery system can be used.

The drug delivery system can be any device, including an implantable device, which device can be based on, for example, mechanical infusion pumps, electromechanical infusion pumps, depots, microspheres. Essentially, and drug delivery system that provides for controlled release as described above (at least biphasic release) is suitable for use in the instant invention. In some embodiments, the drug delivery system is a depot. In other embodiments, the drug delivery system is a continuous delivery device (e.g., an injectable system, a pump, etc.). In still other embodiments, the drug delivery system is a combination of a injection device (e.g., a syringe and needle) and a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

In some embodiments, the delivery system is a depot system. Depot systems comprise a matrix in which the IFN-α or other antiviral agent is embedded. The matrix is a polymeric or non-polymeric substance.

In certain embodiments, drug delivery system comprises a depot.

In some embodiments, the depot comprises a polymeric matrix. For example, a polymeric matrix derived from copolymeric and homopolymeric polyesters having hydrolysable ester linkages may be used. A number of these are known in the art to be biodegradable and to lead to degradation products having no or low toxicity. Non-limiting examples of such polymers are polyglycolic adds (PGA) and polylactic acids (PLA), poly(DL-lactic acid-co-glycolic acid)(DL PLGA), poly(D-lactic acid-coglycolic acid)(D PLGA) and poly(L-lactic acid-co-glycolic acid)(L PLGA). Exemplary ratios for lactic acid and glycolic acid polymers in poly(lactic acid-co-glycolic acid) is in the range of 100:0 (i.e. pure polylactide) to 50:50. Other useful biodegradable or bioerodable polymers include but are not limited to such polymers as poly(ε-caprolactone), poly(ε-caprolactone-CO-lactic add), poly (ε-caprolactone-CO-glycolic acid), poly(β-hydroxy butyric acid), poly(alkyl-2-cyanoacrilate), hydrogels such as poly(hydroxyethyl methacrylate), polyamides, poly(amino acids) (i.e. L-leucine, glutamic acid, L-aspartic acid and the like), poly (ester urea), poly (2-hydroxyethyl DL-aspartamide), polyacetal polymers, polyorthoesters, polycarbonate, polymaleamides, polysaccharides and copolymers thereof.

In some embodiments, the drug delivery system is a poly (lactic acid-co-glycolic acid) system. Such systems are described in the literature, e.g., in U.S. Pat. Nos. 6,183,781; and 5,654,008.

In some of these embodiments, the depot is a high viscosity liquid such as a non-polymeric non-water-soluble liquid carrier material, e.g., Sucrose Acetate Isobutyrate (SAIB) or another compound such as a compound described in U.S. Pat. Nos. 5,968,542; and 5,747,058. For example, the SABER™ system (Southern Biosystems, Inc.) is used.

Release modifying agents and/or additives can be included in the depot matrix.

The term “release modifying agent”, as used herein, refers to a material which, when incorporated into a polymer/drug matrix, modifies the drug-release characteristics of the matrix. A release modifying agent can, for example, either decrease or increase the rate of drug release from the matrix. One group of release modifying agents includes metal-containing salts.

One category of additives includes biodegradable polymers and oligomers. The polymers can be used to alter the release profile of the substance to be delivered, to add integrity to the composition, or to otherwise modify the properties of the composition. Non-limiting examples of suitable biodegradable polymers and oligomers include: poly(lactide), poly(lactide-co-glycolide), poly(glycolide), poly(caprolactone), polyamides, polyanhydrides, polyamino acids, polyorthoesters, polycyanoacrylates, poly(phosphazines), poly(phosphoesters), polyesteramides, polydioxanones, polyacetals, polyketals, polycarbonates, polyorthocarbonates, degradable polyurethanes, polyhydroxybuty-ates, polyhydroxyvalerates, polyalkylene oxalates, polyalkylene succinates, poly(malic acid), chitin, chitosan, and copolymers, terpolymers, oxidized cellulose, or combinations or mixtures of the above materials.

Examples of poly(α-hydroxy acid)s include poly(glycolic acid), poly(DL-lactic acid) and poly(L-lactic acid), and their copolymers. Examples of polylactones include poly(ε-caprolactone), poly(δ-valerolactone) and poly(γ-butyrolactone).

Other additives include non-biodegradable polymers. Non-limiting examples of non-erodible polymers which can be used as additives include: polyacrylates, ethylene-vinyl acetate polymers, cellulose and cellulose derivatives, acyl substituted cellulose acetates and derivatives thereof, non-erodible polyurethanes, polystyrenes, polyvinyl chloride, polyvinyl fluoride, poly(vinyl imidazole), chlorosulphonated polyolefins, and polyethylene oxide.

A further class of additives which can be used in the present compositions are natural and synthetic oils and fats. Oils derived from animals or from plant seeds of nuts typically include glycerides of the fatty acids, chiefly oleic, palmitic, stearic, and linolenic.

Other additives include film property modifying agents and release controlling agents. Examples of film property modifying agents include plasticizers, e.g. triethyl-citrate, triacetin, polyethyleneglycol, polyethyleneoxide etc. Examples of release-controlling agents include inorganic bases (e.g. sodium hydroxide, potassium hydroxide, sodium carbonate, potassium carbonate, etc), organic bases (e.g. ethanol amine, diethanole amine, triethanole amine, lidocaine, tetracaine, etc,), inorganic acids (e.g. ammonium sulfate, ammonium chloride, etc), organic acids (e.g. citric acid, lactic acid, glycolic acid, ascorbic acid, etc), and solid soluble substances which upon release create pores in the coating (e.g. crystals of sodium chloride, glucose, mannitol, sucrose, etc).

In some embodiments, the drug delivery system is a polyethylene glycol-poly(lactic co-glycolic) acid (PEG-PLGA) based aqueous injectible thermosensitive gel, as described in, e.g., U.S. Pat. Nos. 6,201,071; 6,117,949; and 6,004,573. For example, the depot can comprise a water soluble, biodegradable ABA- or BAB-type tri-block polymer is disclosed that is made up of a major amount of a hydrophobic A polymer block made of a biodegradable polyester and aminor amount of a hydrophilic PEG B polymer block, having an overall average molecular weight of between about 2000 and 4990, and that possesses reverse thermal gelation properties. Such materials form a gel depot within the body, from which the drugs are released at a controlled rate.

In some embodiments, the drug delivery system is a polyamino acid-based system, e.g., as described in U.S. Pat. Nos. 6,071,538; 6,245,359; 6,221,367; and 6,099,856.

In other embodiments, the drug delivery system is a microsphere. Microspheres are amply described in the literature.

In another embodiments, the drug delivery system is a pump, e.g., an implantable pump, particularly an adjustable implantable pump. Of particular interest is the use of an adjustable pump, particularly a pump that is adjustable while in position for delivery (e.g., externally adjustable from outside the patient's body. Such pumps include programmable pumps that are capable of providing high concentrations of IFN-α or other antiviral agent over extended periods of time, e.g., 24-72 hours, and to achieve AUC serum IFN-α concentrations to be therapeutically effective.

In some embodiments, the delivery device is a Medipad® device (Elan Pharm Int'l. Ltd.).

Mechanical or electromechanical infusion pumps can also be suitable for use with the present invention. Examples of such devices include those described in, for example, U.S. Pat. Nos. 4,692,147; 4,360,019; 4,487,603; 4,360,019; 4,725,852, and the like. In general, the present methods of drug delivery can be accomplished using any of a variety of refillable, pump systems. Pumps provide consistent, controlled release over time.

In a preferred embodiment, the drug delivery system is an at least partially implantable device. The implantable device can be implanted at any suitable implantation site using methods and devices well known in the art. An implantation site is a site within the body of a subject at which a drug delivery device is introduced and positioned. Implantation sites include, but are not necessarily limited to a subdermal, subcutaneous, intramuscular, or other suitable site within a subject's body. Subcutaneous implantation sites are generally preferred because of convenience in implantation and removal of the drug delivery device.

As noted above, a combination of delivery systems can be used. As one non-limiting example, a PLGA based system which has an initial drug release or burst characteristic is combined with a sucrose acetate isobutyrate based system with no drug release as a burst may be combined together to achieve the desired profiles taught by this invention. As another non-limiting example, a loading dose such as a bolus followed by a zero-order throughput as realized or achieved with a device system. The delivery molecule may be an alpha interferon or a PEG derivatized alpha interferon with all these delivery systems.

Depending on the drug delivery system, IFN-α can be administered orally, subcutaneously, intramuscularly, parenterally, or by other routes such as transdermally, cutaneously, etc. There could be a burst of the drug when administered by such routes e.g., orally except that the drug enters portal circulation as in oral delivery and therefore of utility in targeting the drug to the desired organ, namely liver.

In many embodiments, the IFN-α is delivered subcutaneously.

IFN-α is administered to individuals in a formulation with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

IFN-α can be administered together with (i.e., simultaneously in separate formulations; simultaneously in the same formulation; administered in separate formulations and within about 48 hours, within about 36 hours, within about 24 hours, within about 16 hours, within about 12 hours, within about 8 hours, within about 4 hours, within about 2 hours, within about 1 hour, within about 30 minutes, or within about 15 minutes or less) one or more additional therapeutic agents.

In other embodiments, patients are treated with a combination of IFN-α and ribavirin. Ribavirin, 1-β-D-ribofuranosyl-1H-1,2,4-triazole-3-carboxamide, available from ICN Pharmaceuticals, Inc., Costa Mesa, Calif., is described in the Merck Index, compound No. 8199, Eleventh Edition. Its manufacture and formulation is described in U.S. Pat. No. 4,211,771. The ribavirin may be administered orally in capsule or tablet form in association with the administration of IFN-α. Of course, other types of administration of both medicaments, as they become available are contemplated, such as by nasal spray, transdermally, intravenous, by suppository, by sustained release dosage form, etc. Any form of administration will work so long as the proper dosages are delivered without destroying the active ingredient. If administered, ribavirin is administered in an amount ranging from about 400 mg to about 1200 mg, from about 600 mg to about 1000 mg, or from about 700 to about 900 mg per day.

In some embodiments, the combination therapy comprises IFN-α and IFN-γ. In some of these embodiments, IFN-α and IFN-γ are administered in the same formulation, and are administered simultaneously. In other embodiments, IFN-α and IFN-γ are administered separately, e.g., in separate formulations. In some of these embodiments, IFN-α and IFN-γ are administered separately, and are administered simultaneously. In other embodiments, IFN-α and IFN-γ are administered separately and are administered within about 5 seconds to about 15 seconds, within about 15 seconds to about 30 seconds, within about 30 seconds to about 60 seconds, within about 1 minute to about 5 minutes, within about 5 minutes to about 15 minutes, within about 15 minutes to about 30 minutes, within about 30 minutes to about 60 minutes, within about 1 hour to about 2 hours, within about 2 hours to about 6 hours, within about 6 hours to about 12 hours, within about 12 hours to about 24 hours, or within about 24 hours to about 48 hours of one another.

Determining Effectiveness of Treatment

Whether a subject method is effective in treating a hepatitis virus infection, particularly an HCV infection, can be determined by measuring viral load, or by measuring a parameter associated with HCV infection, including, but not limited to, liver fibrosis.

Viral load can be measured by measuring the titer or level of virus in serum. These methods include, but are not limited to, a quantitative polymerase chain reaction (PCR) and a branched DNA (bDNA) test. For example, quantitative assays for measuring the viral load (titer) of HCV RNA have been developed. Many such assays are available commercially, including a quantitative reverse transcription PCR (RT-PCR) (Amplicor HCV Monitor™, Roche Molecular Systems, New Jersey); and a branched DNA (deoxyribonucleic acid) signal amplification assay (Quantiplex™ HCV RNA Assay (bDNA), Chiron Corp., Emeryville, Calif.). See, e.g., Gretch et al. (1995) Ann. Intern. Med. 123:321-329.

As noted above, whether a subject method is effective in treating a hepatitis virus infection, e.g., an HCV infection, can be determined by measuring a parameter associated with hepatitis virus infection, such as liver fibrosis. Liver fibrosis reduction is determined by analyzing a liver biopsy sample. An analysis of a liver biopsy comprises assessments of two major components: necroinflammation assessed by “grade” as a measure of the severity and ongoing disease activity, and the lesions of fibrosis and parenchymal or vascular remodeling as assessed by “stage” as being reflective of long-term disease progression. See, e.g., Brunt (2000) Hepatol. 31:241-246; and METAVIR (1994) Hepatology 20:15-20. Based on analysis of the liver biopsy, a score is assigned. A number of standardized scoring systems exist which provide a quantitative assessment of the degree and severity of fibrosis. These include the METAVIR, Knodell, Scheuer, Ludwig, and Ishak scoring systems.

Serum markers of liver fibrosis can also be measured as an indication of the efficacy of a subject treatment method. Serum markers of liver fibrosis include, but are not limited to, hyaluronate, N-terminal procollagen III peptide, 7S domain of type IV collagen, C-terminal procollagen I peptide, and laminin. Additional biochemical markers of liver fibrosis include α-2-macroglobulin, haptoglobin, gamma globulin, apolipoprotein A, and gamma glutamyl transpeptidase.

As one non-limiting example, levels of serum alanine aminotransferase (ALT) are measured, using standard assays. In general, an ALT level of less than about 45 international units per milliliter serum is considered normal. In some embodiments, an effective amount of IFNα is an amount effective to reduce ALT levels to less than about 45 IU/ml serum.

Methods of Treating Liver Fibrosis

The present invention provides methods of treating liver fibrosis. The methods involve administering an antiviral agent, as describe above, wherein viral load is reduced in the individual, and wherein liver fibrosis is treated. Treating liver fibrosis includes reducing the risk that liver fibrosis will occur; reducing a symptom associated with liver fibrosis; and increasing liver function.

Whether treatment with antiviral agent as described herein is effective in reducing liver fibrosis is determined by any of a number of well-established techniques for measuring liver fibrosis and liver function. Liver fibrosis reduction is determined by analyzing a liver biopsy sample. An analysis of a liver biopsy comprises assessments of two major components: necroinflammation assessed by “grade” as a measure of the severity and ongoing disease activity, and the lesions of fibrosis and parenchymal or vascular remodeling as assessed by “stage” as being reflective of long-term disease progression. See, e.g., Brunt (2000) Hepatol. 31:241-246; and METAVIR (1994) Hepatology 20:15-20. Based on analysis of the liver biopsy, a score is assigned. A number of standardized scoring systems exist which provide a quantitative assessment of the degree and severity of fibrosis. These include the METAVIR, Knodell, Scheuer, Ludwig, and Ishak scoring systems.

The METAVIR scoring system is based on an analysis of various features of a liver biopsy, including fibrosis (portal fibrosis, centrilobular fibrosis, and cirrhosis); necrosis (piecemeal and lobular necrosis, acidophilic retraction, and ballooning degeneration); inflammation (portal tract inflammation, portal lymphoid aggregates, and distribution of portal inflammation); bile duct changes; and the Knodell index (scores of periportal necrosis, lobular necrosis, portal inflammation, fibrosis, and overall disease activity). The definitions of each stage in the METAVIR system are as follows: score: 0, no fibrosis; score: 1, stellate enlargement of portal tract but without septa formation; score: 2, enlargement of portal tract with rare septa formation; score: 3, numerous septa without cirrhosis; and score: 4, cirrhosis.

Knodell's scoring system, also called the Hepatitis Activity Index, classifies specimens based on scores in four categories of histologic features: I. Periportal and/or bridging necrosis; 11. Intralobular degeneration and focal necrosis; 111. Portal inflammation; and IV. Fibrosis. In the Knodell staging system, scores are as follows: score: 0, no fibrosis; score: I, mild fibrosis (fibrous portal expansion); score: 2, moderate fibrosis; score: 3, severe fibrosis (bridging fibrosis); and score: 4, cirrhosis. The higher the score, the more severe the liver tissue damage. Knodell (1981) Hepatol. 1:431.

In the Scheuer scoring system scores are as follows: score: 0, no fibrosis; score: 1, enlarged, fibrotic portal tracts; score: 2, periportal or portal-portal septa, but intact architecture; score: 3, fibrosis with architectural distortion, but no obvious cirrhosis; score: 4, probable or definite cirrhosis. Scheuer (1991) J. Hepatol. 13:372.

The Ishak scoring system is described in Ishak (1995) J. Hepatol. 22:696-699. Stage O, No fibrosis; Stage 1, Fibrous expansion of some portal areas, with or without short fibrous septa; stage 2, Fibrous expansion of most portal areas, with or without short fibrous septa; stage 3, Fibrous expansion of most portal areas with occasional portal to portal (P-P) bridging; stage 4, Fibrous expansion of portal areas with marked bridging (P-P) as well as portal-central (P-C); stage 5, Marked bridging (P-P and/or P-C) with occasional nodules (incomplete cirrhosis); stage 6, Cirrhosis, probable or definite. The benefit of anti-fibrotic therapy can also be measured and assessed by using the Child-Pugh scoring system which comprises a multicomponent point system based upon abnormalities in serum bilirubin level, serum albumin level, prothrombin time, the presence and severity of ascites, and the presence and severity of encephalopathy. Based upon the presence and severity of abnormality of these parameters, patients may be placed in one of three categories of increasing severity of clinical disease: A, B, or C.

In some embodiments, a therapeutically effective amount of antiviral agent is an amount of antiviral agent that effects a change of one unit or more in the fibrosis stage based on pre-and post-therapy liver biopsies. In particular embodiments, a therapeutically effective amount of IFN-α and IFN-γ reduces liver fibrosis by at least one unit in the METAVIR, the Knodell, the Scheuer, the Ludwig, or the Ishak scoring system.

Secondary, or indirect, indices of liver function can also be used to evaluate the efficacy of treatment. Morphometric computerized semi-automated assessment of the quantitative degree of liver fibrosis based upon specific staining of collagen and/or serum markers of liver fibrosis can also be measured as an indication of the efficacy of a subject treatment method. Secondary indices of liver function include, but are not limited to, serum transaminase levels, prothrombin time, bilirubin, platelet count, portal pressure, albumin level, and assessment of the Child-Pugh score. An effective amount of antiviral agent is an amount that is effective to increase an index of liver function by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the index of liver function in an untreated individual, or to a placebo-treated individual. Those skilled in the art can readily measure such indices of liver function, using standard assay methods, many of which are commercially available, and are used routinely in clinical settings.

Serum markers of liver fibrosis can also be measured as an indication of the efficacy of a subject treatment method. Serum markers of liver fibrosis include, but are not limited to, hyaluronate, N-terminal procollagen III peptide, 7S domain of type IV collagen, C-terminal procollagen I peptide, and laminin. Additional biochemical markers of liver fibrosis include α-2-macroglobulin, haptoglobin, gamma globulin, apolipoprotein A, and gamma glutamyl transpeptidase.

A therapeutically effective amount of antiviral agent is an amount that is effective to reduce a serum level of a marker of liver fibrosis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the level of the marker in an untreated individual, or to a placebo-treated individual. Those skilled in the art can readily measure such serum markers of liver fibrosis, using standard assay methods, many of which are commercially available, and are used routinely in clinical settings. Methods of measuring serum markers include immunological-based methods, e.g., enzyme-linked immunosorbent assays (ELISA), radioimmunoassays, and the like, using antibody specific for a given serum marker.

Quantitative tests of functional liver reserve can also be used to assess the efficacy of treatment with antiviral agent. These include: indocyanine green clearance (ICG), galactose elimination capacity (GEC), aminopyrine breath test (ABT), antipyrine clearance, monoethylglycine-xylidide (MEG-X) clearance, and caffeine clearance.

As used herein, a “complication associated with cirrhosis of the liver” refers to a disorder that is a sequellae of decompensated liver disease, i.e., or occurs subsequently to and as a result of development of liver fibrosis, and includes, but it not limited to, development of ascites, variceal bleeding, portal hypertension, jaundice, progressive liver insufficiency, encephalopathy, hepatocellular carcinoma, liver failure requiring liver transplantation, and liver-related mortality.

A therapeutically effective amount of antiviral agent is an amount that is effective in reducing the incidence (e.g., the likelihood that an individual will develop) of a disorder associated with cirrhosis of the liver by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to an untreated individual, or to a placebo-treated individual.

Whether treatment with antiviral agent is effective in reducing the incidence of a disorder associated with cirrhosis of the liver can readily be determined by those skilled in the art.

Reduction in liver fibrosis increases liver function. Thus, the invention provides methods for increasing liver function, generally involving administering a therapeutically effective amount of antiviral agent. Liver functions include, but are not limited to, synthesis of proteins such as serum proteins (e.g., albumin, clotting factors, alkaline phosphatase, aminotransferases (e.g., alanine transaminase, aspartate transaminase), 5′-nucleosidase, γ-glutaminyltranspeptidase, etc.), synthesis of bilirubin, synthesis of cholesterol, and synthesis of bile acids; a liver metabolic function, including, but not limited to, carbohydrate metabolism, amino acid and ammonia metabolism, hormone metabolism, and lipid metabolism; detoxification of exogenous drugs; a hemodynamic function, including splanchnic and portal hemodynamics; and the like.

Whether a liver function is increased is readily ascertainable by those skilled in the art, using well-established tests of liver function. Thus, synthesis of markers of liver function such as albumin, alkaline phosphatase, alanine transaminase, aspartate transaminase, bilirubin, and the like, can be assessed by measuring the level of these markers in the serum, using standard immunological and enzymatic assays. Splanchnic circulation and portal hemodynamics can be measured by portal wedge pressure and/or resistance using standard methods. Metabolic functions can be measured by measuring the level of ammonia in the serum.

Whether serum proteins normally secreted by the liver are in the normal range can be determined by measuring the levels of such proteins, using standard immunological and enzymatic assays. Those skilled in the art know the normal ranges for such serum proteins. The following are non-limiting examples. The normal range of alanine transaminase is from about 7 to about 56 units per liter of serum. The normal range of aspartate transaminase is from about 5 to about 40 units per liter of serum. Bilirubin is measured using standard assays. Normal bilirubin levels are usually less than about 1.2 mg/dL. Serum albumin levels are measured using standard assays. Normal levels of serum albumin are in the range of from about 35 to about 55 g/L. Prolongation of prothrombin time is measured using standard assays. Normal prothrombin time is less than about 4 seconds longer than control.

A therapeutically effective amount of antiviral agent is one that is effective to increase liver function by at least about 10%, at least about 20%, at least about 30%, at least about 40%, at least about 50%, at least about 60%, at least about 70%, at least about 80%, or more. For example, a therapeutically effective amount of antiviral agent is an amount effective to reduce an elevated level of a serum marker of liver function by at least about 10%, at least about 20%, at least about 30%, at least about 40%, at least about 50%, at least about 60%, at least about 70%, at least about 80%, or more, or to reduce the level of the serum marker of liver function to within a normal range. A therapeutically effective amount of IFN-γ is also an amount effective to increase a reduced level of a serum marker of liver function by at least about 10%, at least about 20%, at least about 30%, at least about 40%, at least about 50%, at least about 60%, at least about 70%, at least about 80%, or more, or to increase the level of the serum marker of liver function to within a normal range.

Method of Reducing Risk of Hepatic Cancer

The present invention provides methods of reducing the risk that an individual will develop hepatic cancer. The methods involve administering an antiviral agent, as describe above, wherein viral load is reduced in the individual, and wherein the risk that the individual will develop hepatic cancer is reduced. An effective amount of antiviral agent is one that reduces the risk of hepatic cancer by at least about 10%, at least about 20%, at least about 25%, at least about 30%, at least about 35%, at least about 40%, at least about 45%, at least about 50%, at least about 55%, at least about 60%, at least about 65%, at least about 70%, or more. Whether the risk of hepatic cancer is reduced can be determined in, e.g., study groups, where individuals treated according to the methods of the invention have reduced incidence of hepatic cancer.

Subjects Suitable for Treatment

Individuals who have been clinically diagnosed as infected with a hepatitis virus, particularly HCV, are suitable for treatment with the methods of the instant invention. Individuals who are infected with HCV are identified as having HCV RNA in their blood, and/or having anti-HCV antibody in their serum. Such individuals include naive individuals (e.g., individuals not previously treated for HCV) and individuals who have failed prior treatment for HCV (“treatment failure” patients). Treatment failure patients include non-responders (e.g., individuals in whom the HCV titer was not significantly or sufficiently reduced by a previous treatment for HCV); and relapsers (e.g., individuals who were previously treated for HCV, whose HCV titer decreased, and subsequently increased). In particular embodiments of interest, individuals have an HCV titer of at least about 10⁵, at least about 5×10⁵, or at least about 10⁶, genome copies of HCV per milliliter of serum.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

An individual presenting with an HCV infection is treated with IFN-α. A typical patient presents with about 10⁵ to 10⁷ genome copies of HCV per milliliter serum. IFN-α is administered in a drug delivery system that includes IFN-α in at a concentration of the amounts of 63-189 μg for release over one week or 126-378 μg for release over two week time periods.

In one series of treatment regimens, IFN-α is administered using a subcutaneous pump to achieve zero order input levels at 40 μg/day infusion of the drug subcutaneously.

The concentration of IFN-α in the serum, as well as the viral titer, are measured at various time points, e.g., 0 hour, 6 hours, 12 hours, 24 hours, 48 hours, 4 days, 7 days, 15 days. The results are shown in FIGS. 6. Similar measurements are continued for a period of six months every month after therapy is discontinued.

### Example 2

IFN-α is administered in a range of from 200 mg to 500 mg in a volume of from about 0.2 to 0.5 ml by subcutaneous injection.

Typical Drug Loadings are as follows: A Drug loading of 0.1% w/w provides for a “burst” or loading dose of 10-50%. Thus, 0.1% (0.1 g/100 g) of a 200 mg dose is 200 μg and 5-50% of that released dose in 1248 hours is 10 μg-100 μg (first order release), with the balance of the dose released in a zero-order fashion over the course of 10-16 days (e.g. −5-10 μg/day).

In another dosing regimen, the drug loading is adjusted and the “burst-controlled” to provide adjusted release profiles: Drug loadings of 0.5% (0.5 g/100 g=0.005) of a 200 mg dose would provide for 1 mg doses and therefore release profiles of as much as 1-month with appropriate control of burst (5-20%) and daily maintenance release profiles of as much as 20 μg/day as needed in a zero order fashion.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method for treating hepatitis C virus infection in an individual, the method comprising:
administering a composition comprising interferon-α (IFN-α) in an amount effective to achieve a first serum concentration of IFN-α that is at least about 80% of the maximum tolerated dose (MTD) within a first period of time of about 24 to 48 hours, followed by a second concentration of IFN-α that is about 50% or less than the MTD, which second concentration is maintained for a second period of time of at least seven days.

2. The method of claim 1, wherein a sustained viral response is achieved.

3. The method of claim 1, further comprising administering IFN-γ for a period of from about 1 day to about 14 days before administration of IFN-α.

4. The method of claim 1, wherein IFN-α is administered in a depot.

5. The method of claim 1, wherein IFN-α is administered by continuous infusion.

6. The method of claim 5, wherein said continuous infusion administration is achieved with a pump.

7. The method of claim 1, wherein IFN-α is administered by a single subcutaneous injection followed by continuous infusion using a pump.

8. A method of treating hepatitis C virus infection in an individual, the method comprising:
administering IFN-α in a dosing regimen comprising a first phase and a second phase, wherein, in the first phase, a first serum concentration of IFN-α is achieved that is at least about 80% of the maximum tolerated dose (MTD) within a first period of time of about 24 hours, wherein in the second phase, the ratio of the highest IFN-α serum concentration to the lowest serum IFN-α concentration, measured over any 24-hour period during the second phase, is less than 3, and wherein the highest concentration of IFN-α during the second phase is about 50% or less than the MTD.

9. The method of claim 8, wherein the ratio of the highest IFN-α serum concentration to the lowest serum IFN-α concentration, measured over any 24-hour period during the second period of time is about 1.

10. A method for treating hepatitis C virus infection in an individual, the method comprising:
administering a composition comprising consensus interferon-α (CIFN) in an amount effective to achieve a first serum concentration of CIFN that is at least about 80% of the maximum tolerated dose (MTD) within a first period of time of about 24 hours, followed by a second concentration of CIFN that is about 50% or less than the MTD, which second concentration is maintained for a second period of time of at least seven days.

11. A method of treating hepatitis C virus infection in an individual, the method comprising:
administering consensus IFN-α (CIFN) in a dosing regimen comprising a first phase and a second phase, wherein, in the first phase, a first serum concentration of CIFN is achieved that is at least about 80% of the maximum tolerated dose (MTD) within a first period of time of about 24 hours, wherein in the second phase, the ratio of the highest CIFN serum concentration to the lowest serum CIFN concentration, measured over any 24-hour period during the second phase, is less than 3, and wherein the highest concentration of CIFN during the second phase is about 50% or less than the MTD.

12. A method of treating hepatitis C virus infection in an individual, the method comprising:
administering IFN-α in a dosing regimen comprising a first phase and a second phase, wherein, in the first phase, a first serum concentration C1max of IFN-α is achieved within a first period of time of about 24 hours, wherein in the second phase, a Csus is achieved that is about 50% of C1max or less, and wherein the area under the curve, defined by IFN-α serum concentration as a function of time, during any 24-hour time period in the second phase is no greater than the area under the curve of day 2 to day 3 as shown in FIG. 2.

13. A method of treating hepatitis C virus infection in an individual, the method comprising:
administering consensus IFN-α (CIFN) in a dosing regimen comprising a first phase and a second phase, wherein, in the first phase, a first serum concentration C1max of CIFN is achieved within a first period of time of about 24 hours, wherein in the second phase, a Csus is achieved that is about 50% of C1max or less, and wherein the area under the curve, defined by CIFN serum concentration as a function of time, during any 24-hour time period in the second phase is no greater than the area under the curve of day 2 to day 3 as shown in FIG. 2.
